(19) United States
(12) Patent Application Publication
(10) Pub. No.: US 2016/0262980 A1
(43) Pub. Date: Sep. 15, 2016

(54) Fluid Transfer Devices

(71) Applicant: CONCEPTOMED AS, Ballstad (NO)

(72) Inventors: Christian MIDE, Ballstad (NO); Marius ANDRESEN, Oslo (NO); Rolf BLOMVÅGNES, Rong (NO); Kevin GEERS, Oslo (NO)

(73) Assignee: Conceptomed AS, Ballstad (NO)

(21) Appl. No.: 15/024,378

(22) PCT Filed: Sep. 19, 2014
(86) PCT No.: PCT/EP2014/070043

§ 371 Date: Mar. 24, 2016

(30) Foreign Application Priority Data
Sep. 26, 2013 (GB) 1317142.6

(51) Int. Cl. A61J 1/20; A61M 5/32; A61J 1/14

(52) U.S. Cl. CPC A61J 1/202; A61J 1/2048; A61J 1/1412; A61M 5/3204; A61M 2005/3104

(57) ABSTRACT

A fluid transfer device (2) comprises a prefilled fluid chamber (4) in communication with a needleless fluid transfer tip, a cap (10) covering the fluid transfer tip, and a disconnecting member (14) operable to move the cap (10) so as to at least partially uncover the fluid transfer tip. The disconnecting member (14) comprises a lever member pivotally mounted to the prefilled fluid chamber (4) with a front surface (16) moveable to push the cap (10) away. The cap (10) can therefore be removed in a one-handed operation without requiring manual manipulation of the cap (10).

The present invention relates to fluid transfer devices, in particular but not exclusively to prefilled fluid transfer devices for use in a medical setting. Such devices may comprise a prefilled fluid chamber and disconnecting means operable to facilitate at least partial removal of a cap so as to open the prefilled fluid chamber.

Devices that are prefilled with an active pharmaceutical ingredient (API) or other fluid used for medical purposes are becoming increasingly popular. Such prefilled devices, such as prefilled syringes, tend to provide ease of administration, better dose accuracy, increased assurance of sterility and more convenience for use—not only by healthcare professionals but also untrained workers and patients themselves. Devices such as prefilled syringes are considered to provide an increase in safety by reducing the risk of dosage errors and contamination. Furthermore, prefilled devices are seen to reduce overfill wastage. For example, when filling a syringe from a vial there is a tendency to overfill the syringe. Prefilled devices may also eliminate the need for glass vials or ampoules.

In relation to dosing accuracy, several studies have documented issues attributed to manually filled syringes. Prefilled syringes, on the other hand, provide healthcare professionals with a safe and effective alternative to manually drawing fluids from multi-dose vials or manually filling flush syringes. Using a prefilled syringe enables the required dose to be delivered precisely. Only trace amounts of an API or other fluid will remain in the needle of a prefilled syringe after injection. In contrast, single-use or multi-use vials require the syringe to be overfilled with fluid to ensure that an accurate dose is pulled into the syringe each time.

In terms of contamination, prefilled devices can eliminate potential for multi-dose cross-contamination. It has been found that manually prepared syringes are often contaminated prior to administration, for example flush syringes. It has been documented that flush syringe preparation on hospital wards can result in contamination of intravenous (IV) infusion delivery systems. Studies have shown that patients are often exposed to bloodstream infections from poorly maintained IV lines. Prefilled flush syringes have been found to reduce the risk of catheter-related bloodstream infections.

In terms of convenience, prefilled devices such as prefilled syringes can enable quicker administration and may find use e.g. in emergency situations. The use of prefilled devices is particularly appreciated by patients who need to self-inject. For example, the average time taken to prepare an injection by reconstituting a pharmaceutical product and drawing it into a syringe may be compared to a greatly reduced administration time using a prefilled syringe. A self-injecting patient may save many hours of time over the course of a year. The convenience of prefilled devices also extends to international vaccination programmes. According to the World Health Organisation, the use of multi-does vials to fill syringes often leads to 50% of a vaccine being wasted, or children being turned away because health workers are reluctant to open a new vial just for one child. Prefilled injection devices for administering a vaccine may simply comprise a single dose pouch and a needle so that there is no risk of syringe re-use. Such prefilled devices can be so simple to use that vaccines can even be administered by untrained workers.

Prefilled devices such as prefilled syringes may be provided with a needle e.g. for intramuscular and subcutaneous injections, or they may be needleless e.g. when connected to a catheter or IV port to provide continuous therapy and/or flushing. For example, prefilled syringes of saline solution or heparin are often used to flush a catheter before/after drug administration. Other prefilled devices may find use in wound cleaning, eye washing, etc.

Typically a prefilled device such as a prefilled syringe has its fluid chamber sealed and closed by a cap. It is important for the device to stay sterile and the contents not to be leaked. Normally the cap is connected by a screw fit and a user therefore needs two hands in order to hold the device while unscrewing the cap. This manual operation brings a user's hand close to the fluid transfer tip and may result in contamination of the previously sterile device. Where the device is being used by a patient, the patient may lack the strength and/or manual dexterity required to unscrew the cap.

The present invention seeks to address or at least mitigate the problems outlined above.

According to a first aspect of the present invention there is provided a fluid transfer device comprising a prefilled fluid chamber in communication with a needleless fluid transfer tip, a cap covering the fluid transfer tip, and a disconnecting member operable to move the cap so as to at least partially uncover the fluid transfer tip.

It will be appreciated that providing a fluid transfer device with its own dedicated disconnecting member means that the cap can be moved to uncover the fluid transfer tip without requiring manual manipulation of the cap. Operation of the disconnecting member may allow for more controlled movement or removal of the cap. The disconnecting member may allow a single-handed operation as opposed to needing two hands to untwist the cap from the tip. Such a device may enable the cap to be removed more quickly and efficiently from covering the fluid transfer tip. Furthermore, the disconnecting member may be easier for a user to operate so that less manual dexterity is required.

The device may find a wide variety of uses, ranging from fluid transfer into a catheter or IV port, for the purposes of therapeutic infusion and/or flushing, to cuff inflation, and to wound cleaning or other washing purposes. Accordingly the prefilled fluid chamber may take the form of a syringe barrel, fluid delivery pipe or hose, single or multi-dose fluid pouch, etc. The prefilled fluid chamber may be rigid (e.g. a syringe barrel) or flexible (e.g. a squeezable pouch). The fluid being transferred may comprise liquid and/or gas. The fluid may be a liquid, cream, gel, emulsion, etc. The chamber may be prefilled with fluid such as one or more active pharmaceutical ingredients (API), saline, electrolyte solution, heparin or other anti-coagulant, water, silicone, or any other fluid to be transferred for medical or therapeutic purposes.

Preferably the disconnecting member is manually operable to move the cap and at least partially uncover the fluid transfer tip. While one hand may be used to operate the disconnecting member while another holds the device, preferably the disconnecting member is moveably mounted to the prefilled fluid chamber so that a user of the device can operate the disconnecting member at the same time as handling the fluid chamber.

As is mentioned above, it is preferable that operation of the disconnecting member consists of manual movement. The movement of the disconnecting member may take any suitable form. In some embodiments the disconnecting member may be arranged to rotate around the axis of the fluid transfer tip so as to move the cap, for example acting to unscrew the cap. Rotation of the disconnecting member may cause it to move along the fluid transfer tip so as to push away the cap, for example releasing a friction fit or even forcibly releasing a cap from a screw connection. A rotatable disconnecting member may allow single-handed operation, for example where a thumb can be used to rotate the member. However such rotational motion may be less preferred as it may require a user to hold the device in one hand while rotating the disconnecting member with the other.

In a preferred set of embodiments the disconnecting member (or at least its front surface) is arranged to move substantially linearly, rather than rotationally, relative to the fluid transfer tip. This can ensure that the force applied by the disconnecting member to move the cap is substantially linear, or at least without any twisting around the fluid transfer tip. A substantially linear motion of the disconnecting member may include an angular component, preferably a component at an azimuthal angle to the axis of the fluid transfer tip. For example, a pivoting motion may result in substantially linear movement without any rotation around the axis of the fluid transfer tip. Of course, while the disconnecting member is arranged to move linearly along the fluid transfer tip it may be operated as part of a mechanism that includes rotating parts. For example, the disconnecting member may pivot and/or rotate relative to the prefilled fluid chamber. What is important is that a part (e.g. front surface) of the disconnecting member that contacts the cap preferably applies a substantially linear force along the axis of the tip to push the cap away and uncover the tip.

In some embodiments, alternatively or in addition, the disconnecting member may be slidingly mounted to the prefilled fluid chamber with a front surface moveable along the fluid transfer tip to push away the cap. A sliding member provides linear motion and avoids the problems associated with rotational forces as outlined above. The sliding member may have a strip-like rectangular form, e.g. to run along a side of the fluid chamber, or a sleeve-like cylindrical form, e.g. to surround the fluid chamber. As discussed above, the construction of the sliding member may be chosen to ensure its stiffness even if it is formed of a plastics material.

In one set of embodiments, alternatively or in addition, the disconnecting member may be resiliently mounted to the prefilled fluid chamber so as to be biased away from the cap. Applying force to the disconnecting member against the resilient bias may then move the disconnecting member against the cap. The disconnecting member may take the form of a sleeve mounted coaxially with the fluid chamber, for example slidingly mounted as described above, with a resilient means arranged to act against forwards movement of the sleeve.

In one set of embodiments, alternatively or in addition, the disconnecting member may be pivotally mounted to the prefilled fluid chamber. The disconnecting member may be arranged to pivot or swing around the axis of the fluid chamber. As the member pivots, one of its surfaces may move along the fluid transfer tip, especially a cam-like surface. However a pivoting or swivelling motion may not be easy to operate manually, especially with one hand, as it may require a hand to hold the device steady while pivoting the disconnecting member. Preferably the disconnecting member is arranged to pivot so as to move along the axis of the fluid chamber.

Despite the stiffness of the disconnecting member it may still be difficult to transmit enough force to uncap the fluid transfer tip. In a preferred set of embodiments the disconnecting member comprises one or more lever members pivotally mounted to the prefilled fluid chamber with a front surface moveable along the fluid transfer tip to push the cap away. In other words, the disconnecting member may be part of a lever mechanism.

An advantage of using a lever mechanism to uncap the fluid transfer tip is that the lever member(s) can amplify an input force to provide a greater output force, i.e. providing leverage to push the cap away from covering the tip. The mechanical advantage of a lever mechanism can increase the force applied so that the tip can be uncapped using single-handed operation. A lever mechanism therefore represents a particularly preferred configuration for the disconnecting member(s). As before, it is preferable for the lever member(s) to be manually operable.

In order to take advantage of the force amplification provided by a lever mechanism, it is preferable that the lever member(s) are relatively stiff. The lever mechanism may comprise a lever member with a front surface that is substantially transverse to the axis of the fluid transfer tip and one or more side surfaces that extend in a direction substantially parallel to the axis of the tip, the front surface being moveable along the tip to push away the cap.

Each lever member may be pivotally mounted so that its side surfaces lift away from the prefilled fluid chamber when operated. However, in a preferred set of embodiments each lever member is pivotally mounted so that its side surfaces approach the fluid chamber when it is operated. This is because it is generally easier to push down on a lever member than to pull up a lever member. Accordingly a user can operate the lever member by squeezing it against the prefilled fluid chamber. If the fluid chamber is flexible, for example squeezable pouch, then this may also help to transfer fluid out of the chamber. Single-handed operation may therefore be facilitated.

The lever mechanism may comprise multiple lever members, for example a linkage of pivoting lever members. A linkage mechanism may be designed to maximise the mechanical advantage while minimising the range of movement of the mechanism. A linkage mechanism may therefore be ideally suited to small scale fluid transfer devices, where the prefilled fluid chamber has a relatively small volume e.g. 1 ml of fluid.

The member(s) of the lever mechanism may be pivotally mounted so as to be resiliently biased into a position where there is no contact with the cap, or at least no force applied to the cap. For example, a lever member may be mounted with its side surfaces resiliently biased away from the prefilled fluid chamber. This can ensure that the fluid transfer tip is not accidentally uncovered before the fluid is intended to be dispensed. Manual operation of the lever mechanism can then overcome the resilient bias to move the lever member(s) against the cap and push it away from covering the tip.

In any of the embodiments discussed above, the disconnecting member is operable to move the cap so as to at least partially open the prefilled fluid chamber. In some embodiments the disconnecting member may be operable to completely remove the cap from the fluid transfer tip. Alternatively the disconnecting member may be operable to move the cap far enough that the prefilled fluid chamber is opened to atmosphere, e.g. the fluid transfer tip (where provided) is partially uncovered, but the cap is not entirely removed. The disconnecting member may operate to loosen the cap so that it is opened like a valve. Operation of the disconnecting member may control how far the cap moves in the same manner as opening a throttling valve.

Where the prefilled fluid chamber is in communication with a fluid transfer tip, especially a needleless fluid transfer tip, then the cap may remain partially attached to the fluid transfer tip. It is even envisaged that moving the cap may including removing part of the fluid transfer tip that has the cap attached so that a remaining part of the fluid transfer tip is uncovered. One advantage of breaking the fluid transfer tip to move the cap is that the device may be rendered unsuitable for subsequent use e.g. the cap can not be replaced on the tip. This can ensure that medical devices are not re-used and thereby reduce the risk of cross-contamination.

The Applicant has realised that when operating a disconnecting member to move the cap and uncover the fluid transfer tip, especially a lever member that amplifies the input force, then there is a risk of the cap shooting away from the device. This may make it difficult for a user to find the cap and dispose of it. Especially in a medical setting, it is not desirable for loose caps to interfere with patients or procedures, or to roll around the floor and cause a potential hazard.

In a preferred set of embodiments the cap is removably attached to the fluid transfer tip and also further connected to the device. The cap may therefore remain connected to the device even after having been moved to uncover the fluid transfer tip. The cap may even remain connected to the device after having been fully removed from the fluid transfer tip.

This is considered novel and inventive in its own right, and thus when viewed from a second aspect the present invention provides a fluid transfer device comprising a prefilled fluid chamber, a cap arranged to close the prefilled fluid chamber, and a lever member pivotally operable to remove the cap so as to at least partially open the prefilled fluid chamber, wherein the cap is also connected to the device so as to remain attached to the device after having been removed.

The additional connection of the cap to the device will prevent the cap from shooting away from the device upon operation of the lever member. This may be beneficial not only for removing a cap from a needleless device, but also from any device comprising a needle hub attached to the fluid chamber, e.g. via a fluid transfer tip. In at least some embodiments the cap may therefore comprise a needle cap. In some examples the fluid transfer device may comprise a prefilled syringe with a needle hub connected to the fluid chamber provided by the syringe barrel and a cap removably attached to the needle. In other examples the fluid transfer device may comprise a single-dose pouch (filled with a vaccine or the like) in communication with a needle, e.g. integrated with an ampoule, and a cap removably attached to the needle. In other examples the fluid transfer device may comprise an auto injector or pen injector, e.g. for self-administration of a dose of insulin or other medicament.

Such devices carrying a needle may find a wide variety of uses, ranging from infusion into an IV port, to injections such as vaccinations, to connection to a catheter for fluid administration. As before, the prefilled fluid chamber may take the form of a syringe barrel, fluid delivery pipe or hose, single or multi-dose fluid pouch, etc. The prefilled fluid chamber may be rigid (e.g. a syringe barrel) or flexible (e.g. a squeezable pouch). The fluid being transferred may comprise liquid and/or gas. The chamber may be prefilled with fluid such as one or more active pharmaceutical ingredients (API), hormone therapy agents, insulin, or any other pharmaceutical preparation. It will be understood that any reference to a prefilled fluid chamber also includes a chamber that has been refilled with fluid.

Whether or not the device is needleless, the cap advantageously remains attached to the device even after it has been removed to open the fluid chamber. It is often preferable to avoid the re-use of fluid transfer devices, especially those used for medical purposes, but retaining the cap means that it can be replaced if desired. This may be helpful where the device is used to administer multiple doses, for example an auto injector or pen injector. A user will find it easier to remove the cap before each dose with the help of the lever member.

In one set of embodiments the prefilled fluid chamber is in communication with a fluid transfer tip and the cap is arranged to cover the fluid transfer tip. The fluid transfer tip is preferably needleless. While the lever member may operate to only loosen the cap so that fluid can be dispensed through the tip, preferably the lever member is pivotally operable to completely remove the cap from the fluid transfer tip.

The cap may be additionally connected to the device in any suitable way. It may be possible to disconnect the cap completely from the device, e.g. in a further operation, and this may be desirable if, for instance, the cap is to be recycled separately from the rest of the device. However it is preferable that the cap has at least one permanent connection to the device so that it never comes loose.

In one set of embodiments the cap is hingedly connected to the device. The hinge may be closed when the cap is arranged to prevent fluid from leaving the prefilled fluid chamber. The lever member may be operated to open the hinge and swing the cap away so that fluid can leave the prefilled chamber. The cap may be hingedly connected to the prefilled fluid chamber. However this may require a change to the standard design of fluid chambers such as syringe barrels. Instead, the cap may be hingedly connected to the lever member. This means that a fluid chamber such as a syringe barrel having a standard design may be combined with the lever member to provide a novel cap and removal mechanism. The cap may be connected to either the prefilled fluid chamber or the lever member by a living hinge. This may conveniently allow for a single moulding operation when the device is made of a plastics material.

As is mentioned above, removing the cap may not always involve movement of the cap relative to the rest of the device. In some examples, the lever member may operate to remove the cap by removing part of the device that has the cap attached. This has the benefit that the cap can not be replaced so the device can not be re-used. Where the prefilled fluid chamber includes a fluid transfer tip and the cap is attached (permanently or removably) to the fluid transfer tip, then the lever member may operate to remove the fluid transfer tip that has the cap attached. In such cases the fluid transfer tip itself may be hingedly connected to the prefilled fluid chamber. However it may be preferable for the fluid transfer tip to be frangibly connected to the prefilled fluid chamber, so that it can not be restored after the lever member has operated to break the frangible connection.

It will be appreciated that the additional connection provides the benefit of preventing a loose cap regardless of whether a lever member or other disconnection mechanism is provided. Thus, according to a further aspect, the present invention extends to a fluid transfer device comprising a prefilled fluid chamber, a cap arranged to close the prefilled fluid chamber, and a disconnecting member operable to move the cap so as to at least partially open the prefilled fluid chamber, wherein the cap is also connected to the device so as to remain attached to the device after having been moved.

The disconnecting member may comprise one or more disconnecting members having any of the features already discussed hereinabove. The cap may prevent fluid from leaving the prefilled chamber by covering a needless fluid transfer tip, or by covering a needle, that is in fluid communication with the chamber. Such devices may therefore include prefilled flush syringes, prefilled hypodermic syringes, prefilled IV infusion devices, prefilled injection devices (single or multi-dose), prefilled auto injectors or pen injectors, prefilled flushing devices, etc.

There will now be described some general features applicable to embodiments of a fluid transfer device according to any of the foregoing aspects of the invention.

The Applicant has recognised that the material and/or construction of the disconnecting member can be important for controlling the way that a force is applied to move the cap. In particular, it is preferable for the disconnecting member to be relatively stiff so that its operation effectively transmits a force to move the cap. A high force transmission may be required to move the cap, depending on how it is attached e.g. to a fluid transfer tip. For example, the force may need to be large enough to overcome a friction or snap fit, or even to push off a cap that is attached by a screw thread. Ideally the kinetic energy of the disconnecting member is converted efficiently into kinetic energy for the cap to be moved. If the disconnecting member is not stiff then it may deform as it moves and wastefully convert its kinetic energy into potential energy instead. There is a risk that such potential energy may build-up before finally being converted into a burst of kinetic energy that results in the cap shooting away from the device in an uncontrolled fashion, especially where no additional connection is provided to retain the cap. This is highly undesirable, especially in a medical setting, and instead it is preferable for the cap to be moved in a smooth and controlled manner. One solution to this problem could be to form the disconnecting member from an inherently stiff material, for example stainless steel.

In order to provide flexibility in the choice of material for the disconnecting member, another solution is to provide the disconnecting member with a stiff construction. This gives freedom for the disconnecting member to be formed from a plastics material, which may be preferred e.g. for reasons of disposability, recyclability, sterility, ease of manufacture and cost. The Applicant has devised a novel construction in which the disconnecting member has a three-dimensional shape including a front surface arranged to act on the cap and side surface(s) that form a shroud at least partially surrounding the prefilled fluid chamber. In embodiments where the prefilled fluid chamber is in communication with a fluid transfer tip, especially a needless tip, the disconnecting member may comprise a surface that is substantially transverse to the axis of the fluid transfer tip and one or more side surfaces that extend in a direction substantially parallel to the axis of the fluid transfer tip. Preferably the side surfaces form a shroud extending from the tip towards the prefilled fluid chamber. Preferably the side surface(s) form a shroud at least partially surrounding the fluid transfer tip, and further preferably also at least partially surrounding the prefilled fluid chamber. Accordingly it will be understood that the disconnecting member has a three-dimensional construction that is stiffened by the multiple surfaces extending in different directions.

In a set of embodiments the disconnecting member has a substantially cylindrical form with one or more side surface(s) extending substantially transverse to the front surface. In at least one set of embodiments the disconnecting member has a front surface connected to one or more side surfaces that at least partially surround the prefilled fluid chamber. The surrounding side surfaces may have a cylindrical form or any other suitable shape, for example rectangular. This can stiffen the disconnecting member so that the front surface preferably does not flex when pushed against the cap but instead transmits its kinetic energy to move the cap and uncover the tip. Of course the disconnecting member may be formed so as to have a partially cylindrical form. The side surface(s) do not need to extend fully around the circumference.

The Applicant has realised that the disconnecting member may be stiffened by a three-dimensional shape having side surface(s) that form a shroud at least partially surrounding the prefilled fluid chamber, but the side surface(s) may make it more difficult to mount the disconnecting member to a device, in particular where the prefilled fluid chamber is in communication with a fluid transfer tip. In some embodiments the front surface of the three-dimensional disconnecting member may include an aperture to accommodate the fluid transfer tip. Accordingly the front surface may entirely surround the fluid transfer tip. In some embodiments the front surface of the three-dimensional disconnecting member may be bifurcated to accommodate the fluid transfer tip e.g. between two tongues. The front surface may be forked to make it easier to mount the disconnecting member to a device having a fluid transfer tip. So as to stiffen the disconnecting member after it has been mounted to a device, it may further comprise means to connect the two tongues and form a substantially continuous surface surrounding the fluid transfer tip. A latch, plug or tape connection may be moved to connect the two tongues.

A three-dimensional disconnecting member is stiffer than a substantially two-dimensional, e.g. planar, member and may therefore be more effective in transmitting kinetic energy. This can be particularly advantageous if it is desired to form the disconnecting member from a plastics material. The construction of the disconnecting member may be further designed for optimal stiffness. Alternatively, or in addition, the front and side surface(s) of the disconnecting member are preferably integrally formed. For example, at least these parts of the disconnecting member may be formed as a single plastics moulding.

In embodiments where the one or more side surfaces at least partially surround the fluid chamber, for example in the form of a shroud extending back from the front surface, then the disconnecting member can advantageously sit close to the prefilled fluid chamber rather than sticking out. This makes the device more compact for the purposes of packaging, transportation, storage, etc. In particular it is preferable for the disconnecting member to comprise a lever member, as is discussed above, and then the side surface(s) can provide an input part that is moved towards or away from the fluid chamber so as to pivot the lever member and push the front surface against the cap. When the side surface(s) generally surround the prefilled fluid chamber, such a lever member can be easily operated in one hand by squeezing the input part towards the fluid chamber e.g. in a similar manner to squeezing a trigger. Moreover the stiffness provided by the three-dimensional e.g. shroud-like construction ensures that force applied to the input part is efficiently transmitted as pivotal motion that moves the front surface to push the cap.

The fluid transfer device may include means for mounting the disconnecting member. Where the disconnecting member comprises one or more side surfaces that extend in a direction substantially parallel to the prefilled fluid chamber, for example in a cylindrical or rectangular form, the side surface(s) can conveniently extend along at least part of the device to engage with the mounting means. Accordingly the fluid transfer device can be conveniently provided with the disconnecting member mounted ready for assistance in uncapping the tip. Embodiments of the present invention may therefore provide a new category of prefilled fluid transfer devices, such as prefilled syringes, that are manufactured and/or sold with a disconnecting member pre-mounted ready for use. While the disconnecting member could potentially be packaged separately and mounted to a device as required, it is advantageous for the device to be packaged and sold as a single unit comprising the disconnecting member mounted thereto.

In a set of embodiments the disconnecting member is mounted to the prefilled fluid chamber. Where the disconnecting member has a three-dimensional shape, the side surface(s) may extend parallel to the prefilled fluid chamber for mounting purposes. Preferably the side surface(s) form a shroud extending to at least partially surround the prefilled fluid chamber and engage with mounting means provided by the fluid chamber.

In a set of embodiments the disconnecting member is not mounted directly to the device. It is envisaged that a fluid transfer device such as a prefilled syringe may be retrofitted with a disconnecting member (and potentially also a cap), for example where the prefilled fluid chamber is made of glass due to its fluid contents and/or area of use. In such embodiments the disconnecting member may be indirectly mounted to the prefilled fluid chamber e.g. by a connecting collar or other means. In one example the disconnecting member and cap may be mounted together to a prefilled fluid chamber, for instance where it is desired to improve the usability of an existing device.

The Applicant has recognised that it may be desirable for the disconnecting member to be dismounted from the device after use. This may even enable the disconnecting member to be re-used with other devices, optionally after sterilisation to avoid cross-contamination. For example, a user may prefer to use a device such as a prefilled syringe without the disconnecting member being present once the cap has been loosened or removed. In a set of embodiments the disconnecting member is operable to move the cap and to become dismounted from the prefilled fluid chamber. Accordingly a user may release the cap and push off the disconnecting member in a single operation, which may be achieved with a single hand.

It may be desirable for the disconnecting member to be disabled during supply or transportation of a device so that there is no risk of accidental uncapping. In some embodiments the device may comprise means for locking the disconnecting member so that it can not be operated. Such locking means may hold the disconnecting member against the prefilled fluid chamber. The disconnecting member may therefore be locked to provide the device with a compact configuration e.g. for storage, transport, etc. In some embodiments the device may comprise means for blocking operation of the disconnecting member. Such blocking means may hold the disconnecting member away from the prefilled fluid chamber so that it can not be moved towards the fluid chamber to act on the cap until the blocking means is first removed or disabled. The blocking means may take the form of a moveable wedge. Alternatively, or in addition, such blocking means may prevent the disconnecting member from contacting or pushing against the cap. A user may need to apply a sufficient force to overcome the blocking means before the disconnecting member can move the cap.

The disconnecting member may comprise at least one disconnecting member, for example one or more members, operable to move the cap and at least partially open the prefilled fluid chamber. It will be understood that the foregoing description applies regardless of the number of disconnecting members.

The prefilled fluid chamber, including a fluid transfer tip where provided, may be closed by a seal in addition to the cap. The device may include such a seal to ensure the sterility of the fluid. Preferably the seal is automatically broken when the device is used, e.g. by operation of the disconnecting or lever member, or by moving the cap, or by the pressure of fluid being dispensed.

The cap may be arranged to close the prefilled fluid chamber in any suitable way. Where the device includes a fluid transfer tip, the cap is preferably fitted to the tip. The cap and/or tip may be tapered so as to provide a friction fit. Alternatively, or in addition, the cap may have means for positively engaging with the tip. Such positive engagement may comprise a flange or groove to provide a click fit, or a snap fit. The cap may be attached to the device by a screw connection. The disconnecting member may operate to unscrew the cap or to forcibly remove the cap by pushing it over threads of the screw connection.

Where the device includes a fluid transfer tip, the tip may be tapered to provide a friction fit with a corresponding hub e.g. a needle hub or IV port. The fluid transfer tip may be tapered to provide a standard Luer Slip connection. The fluid transfer tip may optionally include a threaded collar to provide a standard Luer Lock connection. Although standard Luer Slip or Luer Lock connections use a male tapered tip that fits inside a female hub, it is envisaged that this could be reversed and the fluid transfer tip could be a female part having an internal taper to form friction fit with a corresponding male hub.

Alternatively, or in addition, the fluid transfer tip may comprise means for gripping a hub in a locked position. This may include a snap-fit connection, latch means, gripping finger, etc. that positively engage i.e. grip a hub when it is connected to the tip. This may be particularly suitable for high pressure fluid connections e.g. when transferring more viscous fluids.

The cap may be manufactured separately from the fluid transfer device and fitted to close the chamber after it has been prefilled. However, in at least some embodiments the cap may be integrally formed with part of the device. As is mentioned above, the cap may be formed with an integral hinged connection to the prefilled fluid chamber or disconnecting member. In some embodiments the prefilled fluid chamber may be in fluid communication with a fluid transfer tip and the cap may be integrally formed with the tip. So as to be able to uncover the tip, the cap may be formed with one or more frangible connections to the tip. Operation of the disconnecting member (e.g. lever member) may apply a force to the cap to break the frangible connection(s) and move the cap far enough to uncover the tip. This may be particularly suitable where operation of the disconnecting member removes the cap completely from the tip. The cap may or may not remain attached to the device by an additional connection.

It will be understood that what is meant by a cap is a closed cover that does not allow any fluid to leave the tip when the cap is associated with the tip. A cap prevents the fluid inside the prefilled chamber from contacting the atmosphere until the cap is moved or removed to uncover the tip. Therefore a cap can be clearly distinguished from a fluid transfer hub comprising a needle or other fluid transfer means allowing fluid to leave or enter the tip.

In some examples the cap may be generally cylindrical in shape. However the cap may instead comprise a generally flat cover or plate. The cap may prevent the escape of fluid by covering over an exit from the prefilled fluid chamber, whether a fluid transfer tip or other exit. In addition, or alternatively, the cap may extend inside such an exit e.g. in the form of a plug. In such embodiments operation of the disconnecting member to move the cap may cause the plug to be loosened. Movement of the cap may only partially open the prefilled fluid chamber, for example leaving a throttled exit. It is envisaged that movement of the cap may even comprise opening an exit valve.

Some preferred embodiments of the present invention will now be described, by way of example only, and with reference to the accompanying drawings, in which:

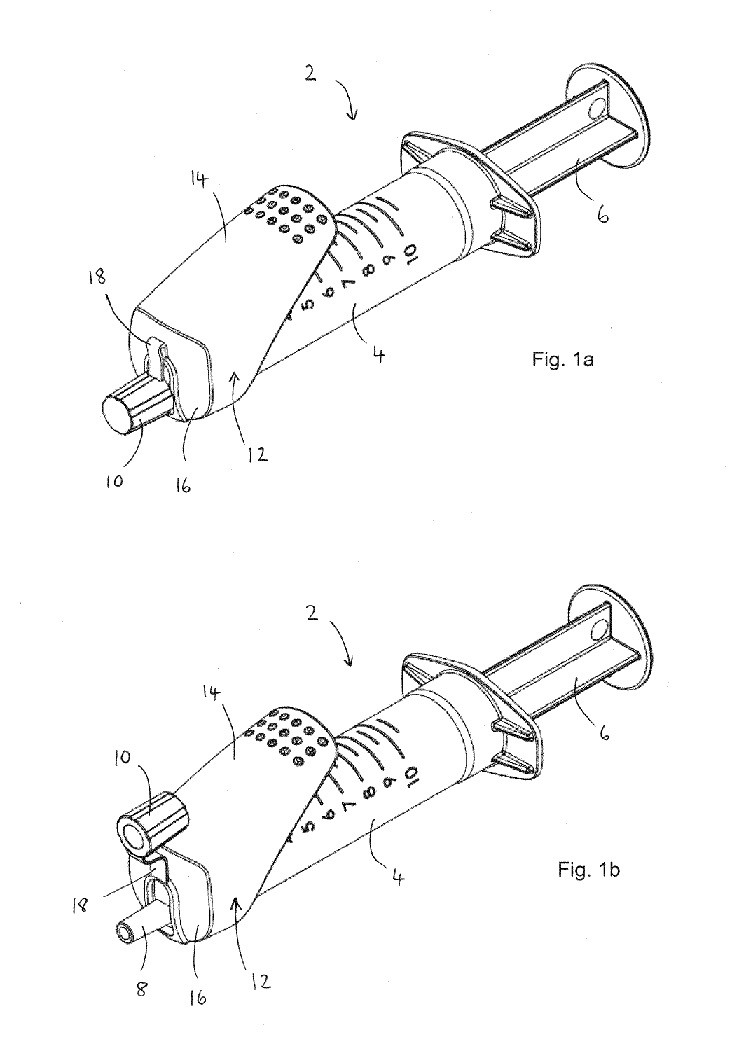

FIG. 1a is a perspective view of a pre-filled syringe that is capped and has a lever member which has not yet been operated;

FIG. 1b is a perspective view of the same pre-filled syringe following operation of the lever member to move the cap;

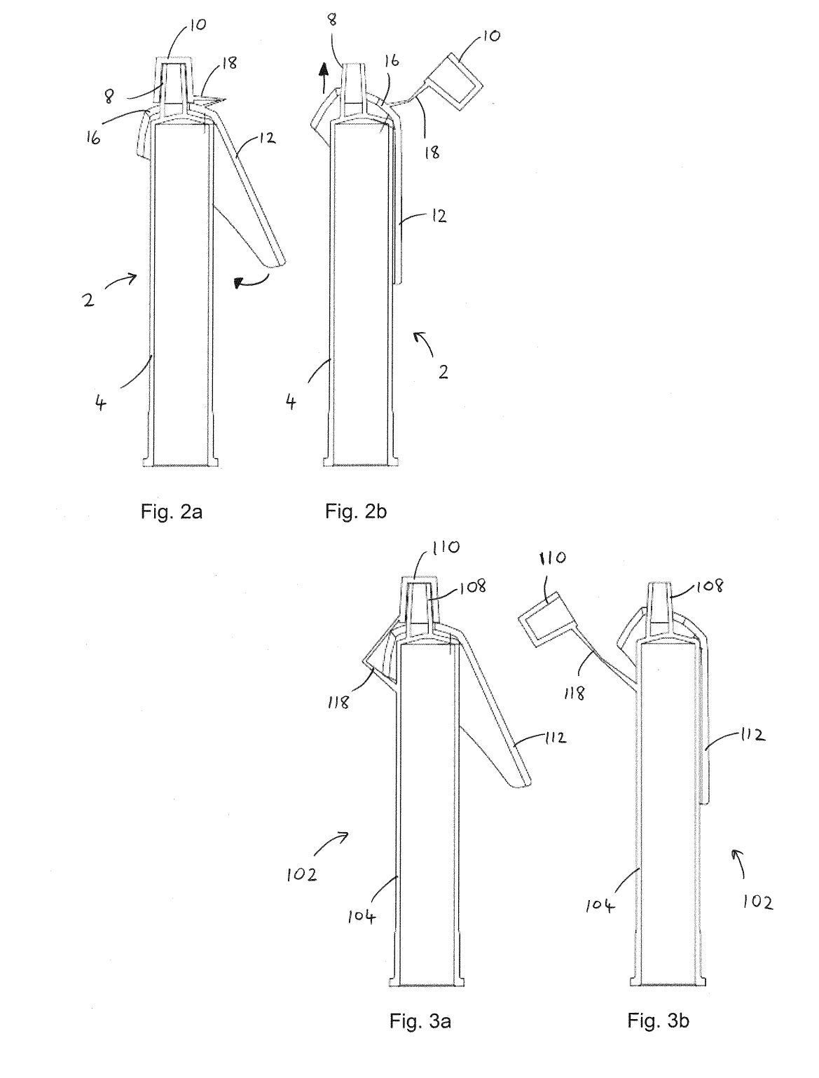

FIGS. 2a and 2b are cross-sectional views of the pre-filled syringe generally corresponding to FIGS. 1a and 1b;

FIGS. 3a and 3b are cross-sectional views of a pre-filled syringe according to an another embodiment;

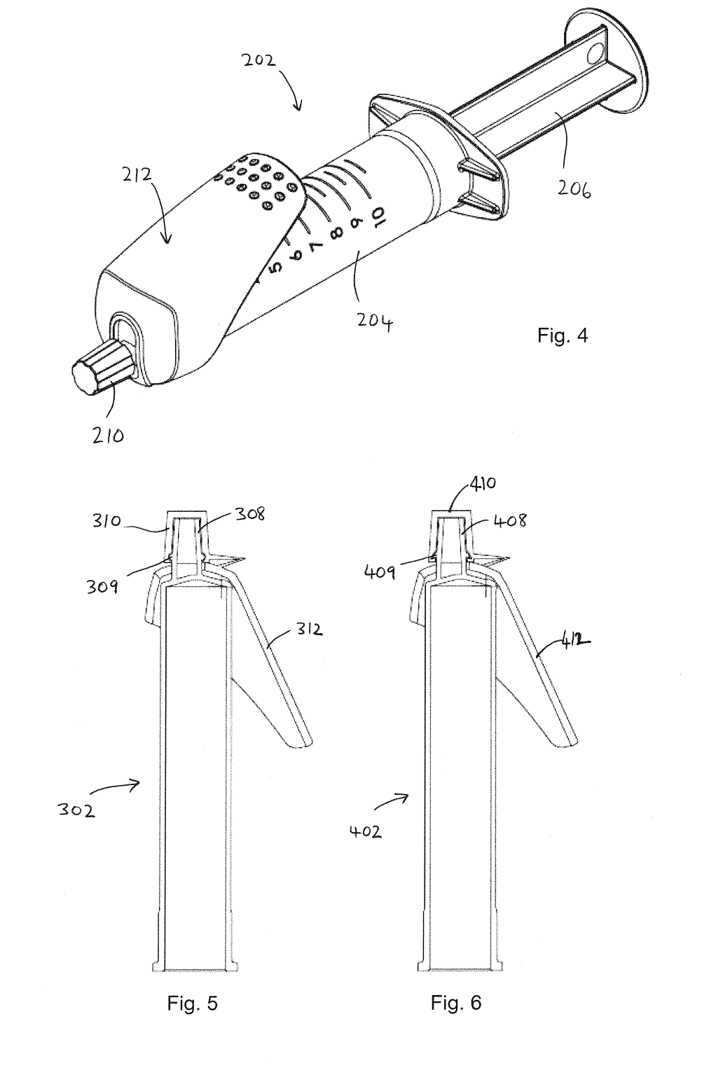

FIG. 4 is a perspective view of a pre-filled syringe according to another embodiment;

FIG. 5 is a cross-sectional view of a pre-filled syringe with a fluid transfer tip having a click-fit;

FIG. 6 is a cross-sectional view of a pre-filled syringe with a fluid transfer tip having a snap-fit;

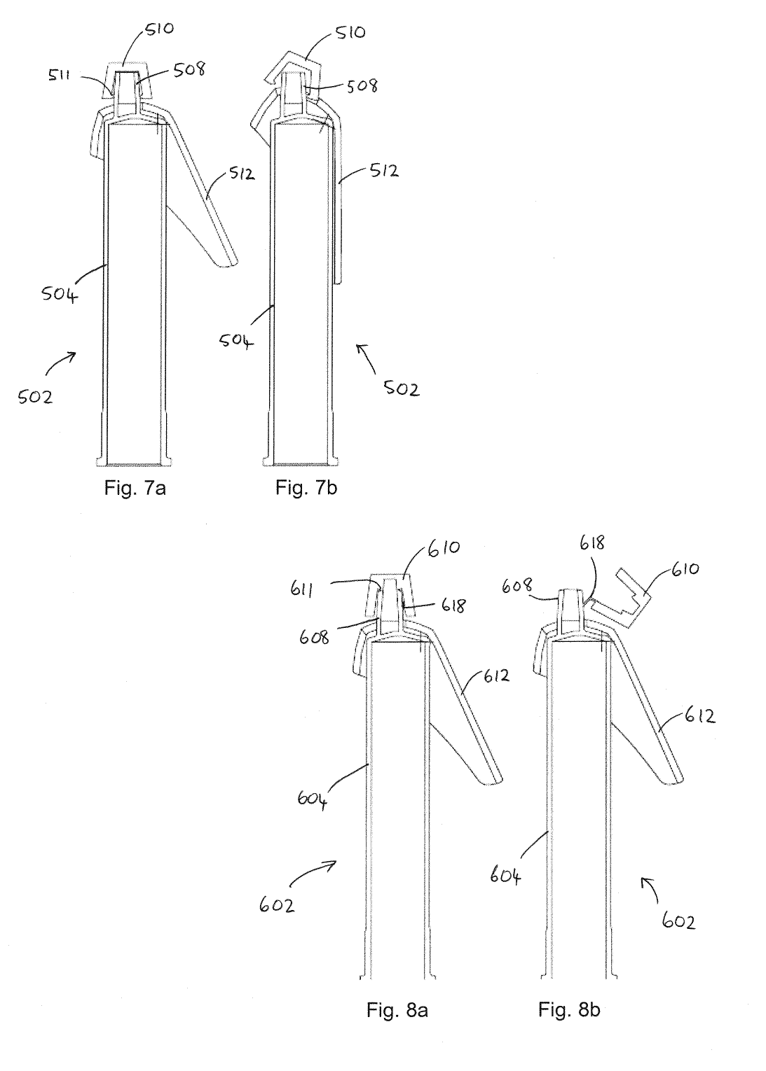

FIGS. 7a and 7b are cross-sectional views of a pre-filled syringe according to a further embodiment;

FIGS. 8a and 8b are cross-sectional views of a pre-filled syringe according to another further embodiment;

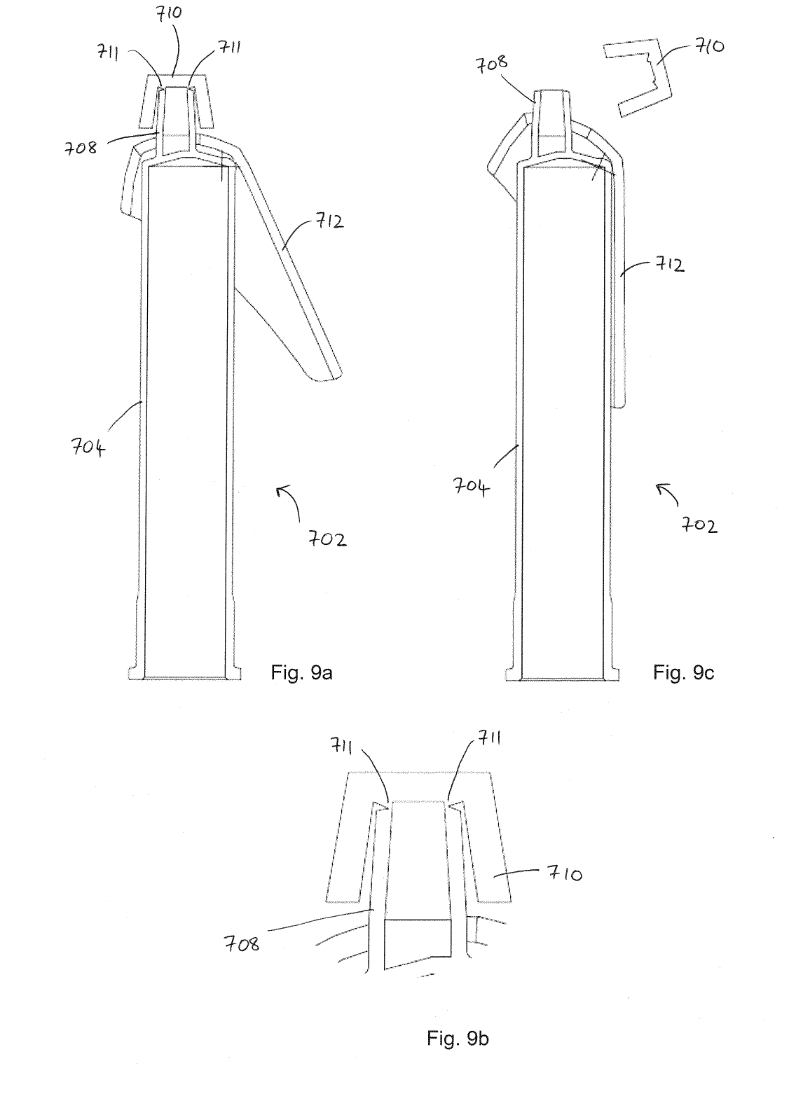

FIGS. 9a to 9c are cross-sectional views of a pre-filled syringe according to a yet further embodiment; and

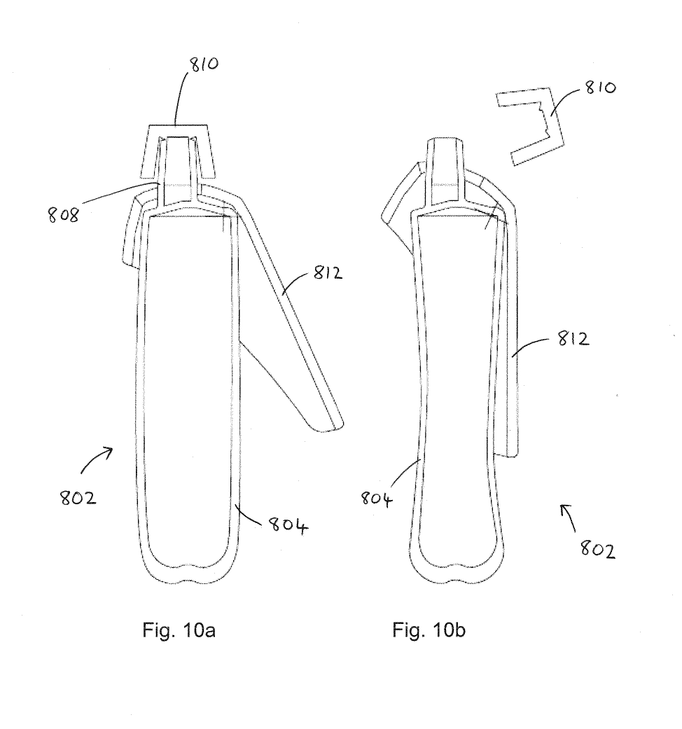

FIGS. 10a and 10b are cross-sectional views of a squeezable fluid transfer container according to an alternative embodiment.

There is seen in FIGS. 1a and 1b a fluid transfer device in the form of a pre-filled syringe 2 comprising a barrel 4 that is pre-filled with fluid such as an active pharmaceutical ingredient and a plunger 6 for administration of the fluid. At the end of the barrel 4 there is provided a fluid transfer tip 8 (seen in FIG. 1b) which can be connected to the inlet port of a fluid delivery system such as a catheter or IV line. It can be seen that the fluid transfer tip 8 is needleless. In this embodiment the fluid transfer tip 8 is tapered so as to provide a friction or interference fit with a corresponding connector. It can be seen from FIG. 1a that the pre-filled syringe 2 is provided as a sterile unit comprising a cap 10 covering the fluid transfer tip 8. The syringe 2 is pre-filled and the cap 10 put in position during manufacture. It will be understood that the pre-filled syringe 2 may include an internal seal in addition to the cap 10, for example a film or foil cover that is broken e.g. when the cap 10 is moved or when the plunger 6 is operated.

It is further seen in FIGS. 1a and 1b that the pre-filled syringe 2 comprises a lever member 12 pivotally mounted to the barrel 4. A side surface 14 of the lever member 12 extends along the barrel 4 to provide a surface that can be pressed down e.g. by a thumb of a user. The lever member 12 also has a front surface 16 that is forked or apertured to accommodate the fluid transfer tip 8. When the lever member 12 is in its rest position as seen in FIG. 1a, the front surface 16 sits behind the cap 10, next to the syringe barrel 4. It is further seen that the cap 10 is additionally connected to the lever member 12 by a hinge 18. The cap 10 and/or hinge 18 may be attached e.g. fused to the lever member 12 during manufacture, or the three parts may be integrally moulded as one piece.

As is seen from FIG. 1b, the lever member 12 may be operated to move the cap 10 and uncover the fluid transfer tip 8. This may be achieved by squeezing a side surface 14 of the lever member 12 down against the syringe barrel 4, so that the front surface 16 rotates forwards along the fluid transfer tip 8 and pushes the cap 10 away. However, because the cap 10 is connected to the lever member 12 by the hinge 18, the cap 10 is not shot free but remains attached to the syringe 2 after having been removed from the fluid transfer tip 8. The lever member 12 may be resiliently biased away from the syringe barrel 4 e.g. by a spring member (not shown) and hence the lever member 12 may return to its starting position as soon as pressure is released after operating the lever member 12, as is seen in FIG. 1b. An advantage of the lever member 12 pivoting back to its starting position is that the front surface 16 moves back down towards the syringe barrel 4 so that it does not interfere with the fluid transfer tip 8 being connected to a fluid transfer system e.g. an IV port of the like. The lever member 12 conveniently enables single-handed operation of the pre-filled syringe 2, with a user simply squeezing the lever member 12 to remove the cap 10 when the syringe 2 is ready to be used. After releasing the lever member 12, the same hand can be used to operate the plunger 6 and therefore transfer fluid out of the syringe 2. In some examples the needleless fluid transfer tip 8 may be used to transfer fluid into a port e.g. of a corresponding connector. In other examples the fluid transfer tip 8 may be connected to a needle hub so that fluid can be injected using a needle.

The cross-sectional views seen in FIGS. 2a and 2b demonstrate how operation of the lever member 12 causes its front surface 16 to move forwards along the fluid transfer tip 8 so as to push off the cap 10. In FIG. 2a the lever member 12 is primed in an initial position with its front surface 16 not contacting the cap 10 or at least not applying any force to the cap 10. Although not shown, a resilient member such as a spring may be provided between the lever member 12 and the barrel 4 of the syringe 2 to bias the lever member 12 into this position. As is seen from FIG. 2b, when the lever member 12 is pressed down against the syringe barrel 4, optionally against the bias of a spring member, this pivoting motion results in the front surface 16 of the lever member 12 moving substantially linearly along the fluid transfer tip 8 and releasing the cap 10 from its friction fit. The hinged connection 18 means that the cap 10 remains attached to the device 2 even after it has been removed from the fluid transfer tip 8.

The cross-sectional views of FIGS. 3a and 3b show an alternative pre-filled syringe 102. In this embodiment the cap 110 has an additional hinged connection 118 to the syringe barrel 104 rather than to the lever member 112. The lever member 112 operates in the same way to remove the cap 110 from the needleless fluid transfer tip 108, the only difference being that the cap 110 remains attached to the syringe barrel 104 after it has been removed.

FIG. 4 shows an alternative embodiment for a pre-filled syringe 202 generally having the same features as described above with respect to FIGS. 1 to 3. The syringe 202 also has a pivotally mounted lever member 212 that can be operated e.g. by squeezing the lever member 212 down against the syringe barrel 204 so as to push away a cap 210. In this embodiment the cap 210 may or may not be additionally connected to the syringe 202. Thus, in some examples the lever member 212 may simply operate to uncover the fluid transfer tip covered by the cap 210 and the loosened cap 210 may be free to fall away from the syringe 212. In other examples, the cap 210 may have a hinged connection, or other internal connection, to the fluid transfer tip (not seen) such that the lever member 212 may operate to loosen but not completely remove the cap 210. This may enable fluid to be dispensed from the syringe 202 using the plunger 206 in situations where it is not necessary for the fluid transfer tip to be connected into a corresponding port. For example, once the cap 210 has been loosened a user may simply operate the plunger 206 to squirt out fluid for the purposes of flushing a wound or the like.

In the embodiments described above, a pre-filled syringe generally has a tapered fluid transfer tip that forms a friction fit with a cap. However, it will be appreciated that the cap may be attached using any suitable fitting and a friction fit may be augmented by an additional positive fit, as is illustrated in FIGS. 5 and 6. In the example of FIG. 5, the needleless fluid transfer tip 308 is not only tapered but also has an outer annular flange 309 that locates inside an annular groove provided on the inside of the cap 310 when the cap 310 is connected onto the tip 308. This provides for a positive “click” fit in addition to the friction fit. Of course, it will be appreciated that the flange and groove may be reversed. Otherwise the pre-filled syringe 302 has a lever member 312 that can be operated in the same manner as described above.

In the example of FIG. 6, the needleless fluid transfer tip 408 is provided with an additional snap-fit ring 409 that positively secures the cap 410 in addition to the friction fit of the tapered tip 408. Otherwise, the pre-filled syringe 402 has a lever member 412 that can be operated in the same manner as described above.

In the embodiments described above, the cap has generally been formed separately from the fluid transfer tip of the syringe, even if it is additionally connected to the lever member or to the syringe barrel to prevent it from being completely removed from the device after release from the tip. However, in at least some embodiments it is envisaged that the cap may be integrally moulded with the fluid transfer tip of a pre-filled syringe or other fluid transfer device, with a disconnecting member such as a lever member operable to break off the cap so as to uncover the fluid transfer tip. Some examples are illustrated in FIGS. 7 to 10.

In FIGS. 7a and 7b there are seen cross-sectional views of a pre-filled syringe 502 comprising a fluid pre-filled in a syringe barrel 504 that is in fluid communication with a tapered tip 508. The needleless fluid transfer tip 508 is covered by a cap 510 that is integrally moulded with the tip 508 e.g. in a plastics material so as to have at least one frangible connection 511. The syringe 502 comprises a lever member 512 that may be pressed against the syringe barrel 504, as is seen from FIG. 7b, so as to pivot and force the cap 510 away from the fluid transfer tip 508. The force applied by the pivoting lever member 512 causes the frangible connection 511 to break so that the cap 510 is moved aside to uncover the fluid transfer tip 508. The cap 510 may remain partially connected to the fluid transfer tip 508, as is seen in FIG. 7b, or operation of the lever member 512 may cause the cap 510 to be removed completely and fall away from the device.

In FIGS. 8a and 8b there is seen an alternative embodiment of a pre-filled syringe 602 comprising a pre-filled syringe barrel 604 in fluid communication with a needleless transfer tip 608 that is initially covered by a cap 610. The cap 610 has at least one frangible connection 611 with the fluid transfer tip 608, and an additional hinged connection 618. It can be seen from FIG. 8b that operating and releasing a pivoting lever member 612 causes the cap 610 to be pushed away from the fluid transfer tip 608 by breaking the frangible connection 611. However, the cap 610 is not completely removed from the syringe 602 and remains attached by the hinged connection 618. There is therefore no danger of the cap 610 shooting away from the syringe 602 when the lever member 612 is operated.

There is seen in FIGS. 9a to 9c another embodiment of a pre-filled syringe 702 wherein the cap 710 is integrally moulded with the needleless fluid transfer tip 708 and initially connected by a frangible connection 711 e.g. a breakable ring around the end of the fluid transfer tip 708. The close-up view of FIG. 9b shows the frangible connection 711 surrounding the fluid transfer tip 708. As is seen from FIG. 9c, operation of the pivotally mounted lever member 712 applies a force to push the cap 710 away from the tip 708 and fully break the frangible connection 711. The cap 710 is therefore completely removed from the syringe 702 and may be free to fall away. An advantage of the lever member 712 is that a user is able to remove the cap 710 using a single-handed operation rather than requiring the manual dexterity to unscrew a conventional cap.

Finally, there is seen in FIGS. 10a and 10b another embodiment of a pre-filled fluid transfer device 802 that takes the form of a flexible fluid container 804 in fluid communication with a needleless transfer tip 808 having a cap 810 connected thereto. A lever member 812 is pivotally mounted to the fluid container 804 and can be operated to remove the cap 810 in a similar way to that described above. While the cap 810 is shown in FIG. 10b as being completely removed, it will be appreciated that an additional hinged connection may be provided where it is desirable for the cap to remain attached to the device 802 after having been removed from the tip 808. It can be seen from FIG. 10b that pressing down the lever member 812 may also cause a user to squeeze the fluid container 804 so that fluid is squirted out of the device 802 as soon as the cap 810 has been removed. Such a device 802 may find use in dispensing a fluid for flushing or washing purposes.

Of course various embodiments of the present invention, such as those described above, are not limited to a fluid transfer device in the form of a syringe. The lever member may be pivotally connected to a device such as a blood collection tube, hose connection, catheter, etc. Furthermore, the needleless fluid transfer tip seen in the illustrated embodiments may, in some examples, be connected to a separable needle hub and the lever member could then be operated to move a cap or sheath so as to uncover the needle.

1. A fluid transfer device comprising a prefilled fluid chamber in communication with a needleless fluid transfer tip, a cap covering the fluid transfer tip, and a disconnecting member operable to move the cap so as to at least partially uncover the fluid transfer tip.

2. A fluid transfer device according to claim 1, wherein the disconnecting member comprises one or more lever members pivotally mounted to the prefilled fluid chamber with a front surface moveable to push the cap away.

3. A fluid transfer device according to claim 1, wherein the disconnecting member comprises a surface that is substantially transverse to the axis of the fluid transfer tip and one or more side surfaces that extend in a direction substantially parallel to the axis of the fluid transfer tip.

4. (canceled)

5. (canceled)

6. A fluid transfer device according to claim 1, wherein the disconnecting member is operable to remove the fluid transfer tip that has the cap attached.

7. (canceled)

8. A fluid transfer device according to claim 1, wherein the disconnecting member is operable to completely remove the cap from the fluid transfer tip.

9. (canceled)

10. A fluid transfer device comprising a prefilled fluid chamber, a cap arranged to close the prefilled fluid chamber, and a disconnecting member operable to move the cap so as to at least partially open the prefilled fluid chamber, wherein the cap is also connected to the device so as to remain attached to the device after having been moved.

11-13. (canceled)

14. A fluid transfer device according to claim 10, wherein the disconnecting member is pivotally mounted to the prefilled fluid chamber.

15. A fluid transfer device according to claim 10, wherein the disconnecting member comprises one or more lever members pivotally mounted to the prefilled fluid chamber with a front surface moveable to push the cap away.

16. A fluid transfer device comprising a prefilled fluid chamber, a cap arranged to close the prefilled fluid chamber, and a lever member pivotally operable to remove the cap so as to at least partially open the prefilled fluid chamber, wherein the cap is also connected to the device so as to remain attached to the device after having been removed.

17. A fluid transfer device according to claim 16, wherein the prefilled fluid chamber is in communication with a fluid transfer tip and the cap is arranged to cover the fluid transfer tip.

18. A fluid transfer device according to claim 17, wherein the lever member is pivotally operable to completely remove the cap from the fluid transfer tip.

19. A fluid transfer device according to claim 17, wherein the lever member is operable to remove the fluid transfer tip that has the cap attached.

20-22. (canceled)

23. A fluid transfer device according to claim 10, wherein the cap is arranged to cover a needleless fluid transfer tip in communication with the prefilled fluid chamber.

24. A fluid transfer device according to claim 10, wherein the prefilled fluid chamber comprises a syringe barrel with a needle hub connected thereto and the cap is removably attached to the needle.

25. (canceled)

26. A fluid transfer device according to claim 10, wherein the cap is hingedly connected to the device.

27. (canceled)

28. A fluid transfer device according to claim 26, wherein the cap is connected by a living hinge.

29. A fluid transfer device according to claim 1, wherein the disconnecting/lever member has a three-dimensional shape including side surface(s) that form a shroud at least partially surrounding the prefilled fluid chamber.

30. A fluid transfer device according to claim 1, wherein the disconnecting/lever member is mounted so as to be resiliently biased away from the prefilled fluid chamber.

31. A fluid transfer device according to claim 1, further comprising means for locking the disconnecting/lever member so that it can not be operated.

32. (canceled)

33. (canceled)

34. A fluid transfer device according to claim 1, consisting of one of: a prefilled flush syringe, a prefilled hypodermic syringe, a prefilled IV infusion device, a prefilled injection device, a prefilled auto injector or pen injector, a prefilled flushing device.
